(19) United States
(12) Patent Application Publication
(10) Pub. No.: US 2010/0071107 A1
(43) Pub. Date: Mar. 25, 2010

(54) Visor Hat

(76) Inventors: Melvin Pharis, Trophy Club, TX (US); Sue W. Pharis, Trophy Club, TX (US)

Correspondence Address: WILSON DANIEL SWAYZE, JR., 3804 CLEARWATER CT., PLANO, TX, 75025, US

(21) Appl. No.: 12/235,700

(22) Filed: Sep. 23, 2008

(51) Int. Cl. A61F 9/00; A42C 5/00

(52) U.S. Cl. 2/12; 2/209.3

(57) ABSTRACT

A visor hat to be worn by user may include a band member and a visor member connected to the band member. The band member may be discontinuous and may be adapted to be adjustable to a plurality of sizes. The band member may include a detachable connection, and the band member may include a hook and loop. Only one end of the band member may include a hook and loop, and both ends of the band member may include a hook and loop. The visor hat may include a connection device, and the connection device may include a first support member. The connection device may include a second support member positioned in an opposed and spaced relationship to the first support member.

### FIELD OF THE INVENTION

The present invention relates to hats and to devices to protect the eyes from the sun and more particularly to a visor hat.

### BACKGROUND

The use of headwear or more commonly known as hats is known in the prior art. More specifically, headwear heretofore devised and utilized for the purpose of reducing glare and shielding the eyes of the wearer are known to consist basically of familiar, expected and obvious structural configurations, notwithstanding the myriad of designs encompassed by the crowded prior art which have been developed for the fulfillment of countless objectives and requirements.

Peak or visor caps are widely worn by golfers, baseball players, hunters and farm workers. These caps have a generally hemisherical shell which fits over the upper portion of the head. The lower edge of the shell extends down close to the top of the ears. A visor or peak, attached to the shell lower edge, extends forward normally about two and one-half to four inches, with three inches about average. The visor usually has a width of about six to eight inches, with seven inches about average. The cap shell is usually made of cloth, which can be insulated for warmth, or partially or wholly of an open mesh material for wear in warm climates where ventilation is desirable. The visor is often made of one or more layers of cloth sewn together, with or without an internal sheet of cardboard or other stiffening material.

The purpose of the visor is to protect the wearers face against excessive sunlight which can cause sunburn and harm the eyes. The visor is also intended to protect the face and eyes against rain. While the normal visor generally provides acceptable protection against the described elements there are times when the shell acts as a hindrance instead of an aid to the user. When temperatures rise to a sufficiently high level, the shell may act to further increase the temperature to the head of the user. In these cases is desirable to keep the visor but to eliminate to the extent possible the shell. An additional problem is achieving a proper fit for the head. The users heads generally do not come in predetermined sizes. Furthermore, the proper fit for the head may change from day to day. Depending on the hairstyle of the user, different sizes of hats may be required in order to accommodate the changing hairstyles.

### SUMMARY

A visor hat to be worn by user may include a band member and a visor member connected to the band member.

The band member may be discontinuous and may be adapted to be adjustable to a plurality of sizes.

The band member may include a detachable connection, and the band member may include a hook and loop.

Only one end of the band member may include a hook and loop, and both ends of the band member may include a hook and loop.

The visor hat may include a connection device, and the connection device may include a first support member.

The connection device may include a second support member positioned in an opposed and spaced relationship to the first support member.

### BRIEF DESCRIPTION OF THE DRAWINGS

The invention may be understood by reference to the following description taken in conjunction with the accompanying drawings, in which, like reference numerals identify like elements, and in which:

FIG. 1 illustrates a top view of the visor hat of the present invention;

FIG. 2 illustrates a perspective view of the visor hat of the present invention being worn by user;

FIG. 3 illustrates a connection device for connecting to ends of the band of the visor hat;

FIG. 4 illustrates an alternate connection device;

FIG. 5 illustrates alternative multiple ends of the band of the visor head of the present invention;

FIG. 6 illustrates alternative ends of the band of the visor head of the present invention.

### DETAILED DESCRIPTION

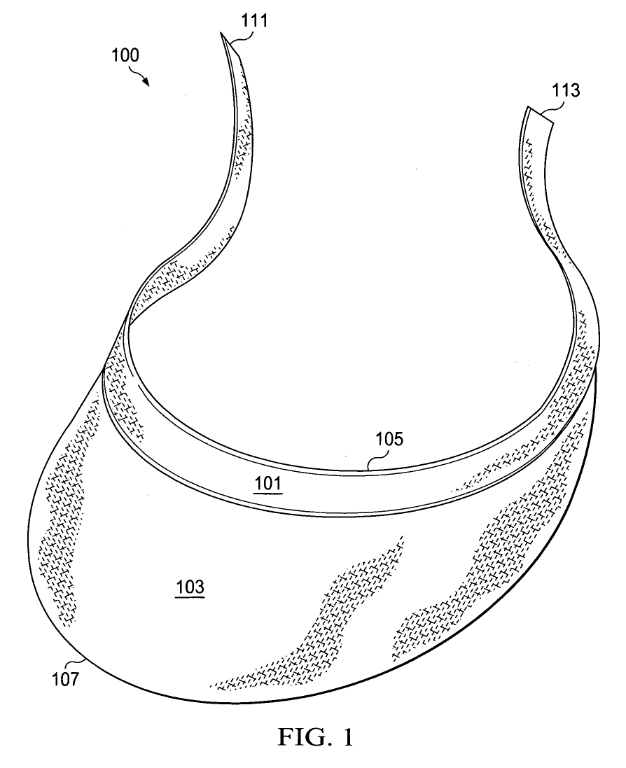

FIG. 1 illustrates a hat visor 100 of the present invention. The hat visor 100 may include a visor member 103 which may be substantially crescent shaped or other suitable shapes. The visor member 103 may include an outer and larger curvature 107 which may be the outside side of the visor member 103 and may include an inner and smaller curvature 105 which may be the inside of the visor member 103 and may be provided on the head contacting side of the user. The hat visor 100 may include a band member 105 which may be a discontinuous band or cord and may be affixed to the inner and smaller curvature 105. The band member 105 may include an elastic member so that the band member 105 may be stretched to different sizes, and the band member 105 may be a continuous band. The band member 105 may extend from a first end 111 of the band member 105 to a second end 113 of the band member 105. The band member 105 and the visor member 103 may be formed from flexible material such as cloth or other suitable material or may be semi rigid. The band member 105 may include a sweatband positioned on a portion of the band member 105 to withdraw perspiration from the head of the user. The visor member 103 may be reinforced by a stiffening member such as cardboard in order to provide a semi rigid member. The band member 105 may be of sufficient length in order that the first end 111 and the second end 113 may be tied together in order to conform to the head of the user. The first end 111 and the second end 113 may be untied and retied in order to change the size of the visor hat 100.

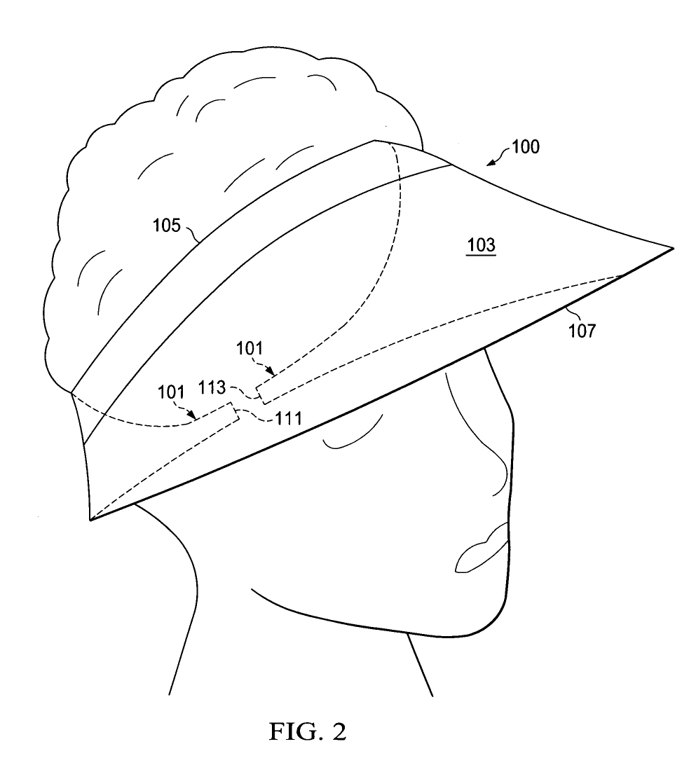

FIG. 2 illustrates a hat visor 100 being worn by a user. The hat visor 100 may include a visor member 103 which may be substantially crescent shaped. The visor member 103 may include an outer and larger curvature 107 which may be the outside side of the visor member 103 and may include an inner and smaller curvature 105 which may be the inside of the visor member 103 and may be provided on the head contacting side of the user. The hat visor 100 may include a band member 105 which may be a discontinuous band or cord and may be affixed to the inner and smaller curvature 105. The band member 105 may include a sweatband positioned on a portion of the band member 105 to withdraw perspiration from the head of the user. The band member 105 may include an elastic member so that the band member 105 may be stretched to different sizes, and the band member 105 may be a continuous band. The band member 105 may extend from a first end 111 of the band member 105 to a second end 113 of the band member 105. The band member 105 may be of sufficient length in order that the first end 111 and the second end 113 may be tied together in order to conform to the head of the user. The first end 111 and the second end 113 may be untied and retied in order to change the size of the visor hat 100.

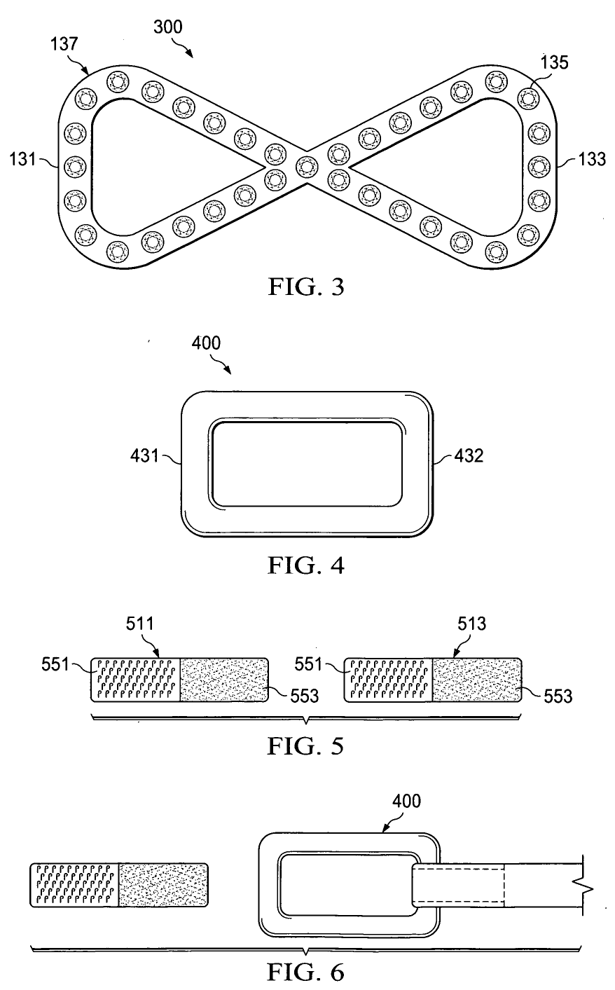

FIG. 3 illustrates a connection device 300 which may be used to detachably connect the first end 111 of the band member 101 to the second end 113 of the band member 101. The connection device 300 may be bowtie or butterfly shaped or other appropriate shape and may include a first support member 131 for connection to the first end 111 and an opposed and spaced second support member 133 for connection to the second end 113. The first support member 131 and the second support member 133 may be connected by a substantially X-shaped and curved connection member 137, and the first support member 131 and the second support member 133 may be positioned substantially traverse to the longitudinal direction of the connection device 300.

FIG. 3 illustrates that the connection device 300 may be decorated with rhinestones 135 or jewels or other decoration devices.

FIG. 4 provides a second connection device 400 which may include a first support member 431 and a second support member 432 which cooperates with the first end 511 and a second end 513 in a similar fashion as described with respect to FIG. 5 and FIG. 3.

FIG. 5 illustrates one alternative for the first end 511 and the second end 513. The first end 511 may include Velcro which may include a hook surface 551 and a loop surface 553 where the hook surface 551 and the loop surface 553 extend around the first support member 131 to detachably connect to the connection device 300 and the second support member 133 where the hook surface 551 engages the loop surface 553 to provide a detachable connection to the connection device 300. The first end 511 and the second end 513 may be connected and disconnected with the Velcro in order to change the size of the visor hat 100.

Consequently, the fit of the band member on the head of the user is adjustable to obtain a multitude of sizes.

FIG. 6 illustrates the second connection device 400 which is affixed to the second end 113 while the first end 511 may include the Velcro including the hook surface 551 and the loop surface 553 in order to detachably connect to the second connection device 400.

The cloth/fabric which may be used for the hat visor may be machine washable with a delicate setting. Some fabrics may require handwashing.

The connection devices or buckles may be sewn into the ends of the band for permanent wear or may be attached or detached to take in consideration the breakable nature of the connection devices or buckles. For example, the above described connection device with rhinestones may be removed prior to washing of the hat visor. Furthermore, the connection device may be attached or detached with ease from the visor.

The hat visor may be packaged by lying it flat or may be rolled up around a soft object. The hat visor should not be folded in order to avoid permanent creases.

The hat visor of the present invention is convenient to use and can be hooked over a belt when not in use. The hat visors can be easily packed.

The ability to interchange the connection device or buckle can make the hat visor flexible enough to be used with informal wear or formal wear, ranging from a wedding to a sporting event.

The visor may be shaped to cover and protect the ears and face from weather or sunlight.

The Velcro including hooks and loops makes the size of the visor hat changeable, allowing the visor to be held in place as tightly as desired.

The visor hat, being of flexible material, results in a soft construction, allowing virtually no pressure against the user which could result in headache or discomfort. The visor hat may be very light weight and fits snug sufficiently to retain the shape in the wind.

The construction of the visor hat may include material of lightweight cottons, twills, cotton and polyester blends or other appropriate material. The band may be formed from the same material as the visor or may be formed from a contrasting fabric in order to complement the overall design.

The band may have a metal ring which may be nickel or brass to connect to one end of the band and may have a hook and loop on the other end of the band. Other bands may have hook and loop on each end of the band to allow the connection device or buckle to be interchanged with another. Consequently, the buckle can be changed to match the attire of the user. Some fabrics are formal and may require a formal buckle.

The visor or brim may include a top and bottom member which may be strengthened by a fusible craft weight bonding which may allow the visor or brim to maintain a predetermined shape and avoid breakdown. Furthermore, top stitching may contribute to the resilience of the brim.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed.

1. A visor hat to be worn by user, comprising:
a band member;
a visor member connected to the band member;
wherein the band member is discontinuous and is adapted to be adjustable to a plurality of sizes.

2. A visor hat to be worn by user as in claim 1, wherein the band member includes a detachable connection.

3. A visor hat to be worn by user as in claim 1, wherein the band member includes a hook and loop.

4. A visor hat to be worn by user as in claim 1, wherein only one end of the band member includes a hook and loop.

5. A visor hat to be worn by user as in claim 1, wherein both ends of the band member include a hook and loop.

6. A visor hat to be worn by user as in claim 1, wherein the visor hat includes a connection device.

7. A visor hat to be worn by user as in claim 6, wherein the connection device includes a first support member.

8. A visor hat to be worn by user as in claim 7, wherein the connection device includes a second support member positioned in an opposed and spaced relationship to the first support member.
